(19) United States
(12) Patent Application Publication
(10) Pub. No.: US 2005/0123879 A1
(43) Pub. Date: Jun. 9, 2005

(54) Device which can be applied in bone and/or tissue in the human body, and method and use of said device

(75) Inventors: Matts Andersson, Lerum (SE); Mikael Eriksson, Molndal (SE)

Correspondence Address: VENABLE, BAETJER, HOWARD AND CIVILETTI, LLP, P.O. BOX 34385, WASHINGTON, DC, 20043-9998, US

(73) Assignee: Nobel Biocare AB, Goteborg (SE)

(21) Appl. No.: 10/500,211

(22) PCT Filed: Dec. 19, 2002
(86) PCT No.: PCT/SE02/02385

(30) Foreign Application Priority Data
Dec. 28, 2001 (SE) 0104446-0

(51) Int. Cl. A61C 8/00; A61F 2/28; B22F 7/06; B22F 3/12; B22F 5/06

(52) U.S. Cl. 433/174; 623/17.17; 623/23.51; 419/19; 419/6

(57) ABSTRACT

A device, for example in the form of an implant, is arranged to be applied via at least one surface or one portion (for example an outer portion) to bone and/or tissue in the human body. An agent which stimulates bone growth, in the form of HA, is used in connection with the device. The device, the surface or the portion comprises or consists of compressed bone-compatible and/or tissue-compatible material in the form of titanium powder. The titanium powder is mixed with the agent, which is also in powder form, and a composite material is formed with the two powders by means of impact compaction. The invention also relates to a method and use in connection with devices of the type in question. A novel type of HA use is made possible and eliminates, inter alia, the disadvantages of loosening HA layers during production of the device.

The present invention relates inter alia to a device which, via at least one surface or one portion, is arranged to be applied in bone and/or tissue in the human body, for example jaw bone. The device is provided, at the surface or portion, with an agent which stimulates bone growth, which can be HA (hydroxyapatite). In addition, at least a part bearing the surface, or the portion, comprises or consists of compressed bone-compatible and/or tissue-compatible powder material, preferably titanium powder. The invention also relates to a method for producing the device in question, which can, for example, be an implant. The invention moreover relates to a use in connection with the production of the device.

It is already known to produce dental crowns, for example, made of titanium powder which is compacted to a great density, for example by a sintering method. In this connection, reference may be made, inter alia, to PCT application WO 00/15137 from the same Applicant as the present patent application. In connection with implants, it is also already known to use a bone-growth-stimulating agent, for example in the form of HA. Reference may be made to the patent literature and inter alia to the patents obtained and the patent applications filed by the same Applicant. In the prior art, it has been proposed to apply HA in layers on the outside of the implant or the like in question. The underlying idea is that the surface layers exposed to the bone or tissue will facilitate the incorporation of the implant or the like.

In connection with the known arrangements and methods, there is a problem in ensuring that the HA layers remain in place, for example during after-treatment of the implant or the like. There is therefore a need for a solution to the problems of the layers coming loose. The main object of the present invention is to solve this problem among others. In accordance with the concept of the invention, a composite material will be created between titanium (Ti) and hydroxyapatite (HA), where the HA is present as particles or fractions admixed in the titanium bulk or the titanium body. By creating a bulk composite, the latter can be used as a raw material for subsequent working of the components in question, without the aforementioned problems of the loosening of the layers of HA. The underlying idea is generally that the HA particles or HA fractions in the surface layer are exposed to the bone and/or tissue and thereby facilitate incorporation of the titanium implant.

In normal pressureless sintering of titanium powder mixed with finely particulate HA powder, these react and form new phases. If a sample sintered in this way is exposed to heat, swelling may occur. There are methods available which are intended to allow these materials to be sintered together without creating any appreciable reactions, but these methods are relatively sophisticated and expensive, for example HIP (hot isostatic pressing) or SPS (spark plasma sintering). There is therefore a need for alternatives to these sintering methods. The invention also has the object of solving these problems.

The feature which can principally be regarded as characterizing the device mentioned in the introduction is that the powder material and the bone-growth-stimulating agent form a composite material which is obtained by means of impact compaction and, if appropriate, subsequent sintering.

In further developments of the inventive concept, the bone-growth-stimulating agent (HA) can be arranged completely or partially in or at the actual surface layer and can thus be exposed to the bone and/or tissue in question. The agent can be chosen with particle sizes or fractions in the range of 90-120 μm. The titanium powder which is used will preferably have a considerable purity, for example a purity of 99.99%, and a relatively small particle size. By way of example, titanium powder in the form of Wah Chang HP (or CP) −325 Mech T080014 (010607) can be included in the composite structure. Titanium powder in a quantity of ca. 90-98%, preferably ca. 95%, and HA powder in a quantity of 2-10%, preferably ca. 5%, form the starting material for the composite material compacted by impaction and possible sintering. The last-mentioned percentage figures are chosen so as to give a total quantity of 100%.

A method according to the invention can be regarded as being characterized principally by the fact that the mixing together of the bone-compatible and/or tissue-compatible powder material and of said agent which is in powder form takes place in a first step. This is followed by application of the mixture in one or more mold cavities belonging to a mold applied in a machine which effects impact compaction and which has properties allowing it to operate with a high impact compaction energy. This is followed by activation of the impacting unit of the machine so that it acts on the mold and transfers the energy to the powder mixture and thereby creates a blank for the device. Finally, the blank is treated in one or more treatment units for producing the device from the blank. In said treatment steps, the blank can be sintered and/or heat-treated and subjected to a treatment or treatments of various types, for example chemical, electrochemical and/or mechanical treatment or machining, for example milling, turning, shot-peening, etc. The machine can be of a kind known per se and is in this case of the type which generates an impact compaction energy of ca. 335 Nm or higher. The machine can operate with one or more impacts against the mold, and the same amounts of energy or different amounts of energy can be used in the different impacts. The titanium particles are compressed to a substantial density, for example 98% or more. The positions of the HA particles in the composite material can be controlled upon mixing and application in the mold cavity of the mold. When the blank is machined to give a finished device or finished surface or finished portion, a desired quantity of HA particles will be present on the surface exposed to the bone and/or tissue in question.

A use according to the invention can be regarded as being principally characterized by the fact that an impact-type compaction machine with a high impact compaction energy is used to compress the powder material and said agent in powder form to give a composite material. By means of what has been proposed above, a device is obtained which is efficient and is simplified from the point of view of use, and a simplified method is obtained. Highly compressed composite bodies can be obtained with the aid of impact compaction (high-velocity compaction). Tests have been carried out on producing a composite material of said type and density, after sintering has been carried out, by cutting up cross sectional surfaces and studying the microstructure and interfaces between titanium and HA.

In said tests, small amounts of the two powders were weighed-in on an analysis balance and mixed in a beaker at 95.00% titanium and 5.00% HA. The powders were mixed in the dry state by brief agitation and stirring.

The powder mixture was impact-compacted at Hydropulsor in Karlskoga in a modified cutting machine “Hydropulver Hyp 30-15”. The powder was placed in a cylindrical, 14-mm press tool of steel lubricated with MOS₂. The powder weight per block was 2.0 g. Five impacts in succession were made against the powder (each block) with 335 Nm energy on each impact. Five such blocks were produced.

The green density was measured with a micrometer screw and with the Archimedes principle in distilled water (without vacuum). Both the measurements gave the same result for the green density. The samples were cut in two in water with a low-speed cut in order to obtain two samples (a+b).

Some of the samples were then heat-treated in vacuum (NB Pp10) in accordance with the following:

| Sample | Ramp ° C./min | Temperature ° C. | Holding time (min) |
|---|---|---|---|
| 1a | 10 | 700 | 60 |
| 1b | 10 | 900 | 6 |
| 2a | 10 | 500 | 600 |
| 2b | Green body | Green body | Green body |
| 3a | — | — | — |
| 3b | — | — | — |
| 4a | — | — | — |
| 4b | — | — | — |
| 5 | — | — | — |

The samples lay on Mo wire on Ti plate in Mo-degel. “Sintered” density was also measured using the Archimedes principle without vacuum directly, after which the samples were dried in a heating chamber at 100° C. for 0.5 h. The densities below may be slightly higher as Ha has a certain porosity which is not taken into calculation.

The results obtained were:

| | Temp./Holding | Green density | Sintered density |
|---|---|---|---|
| Sample | time | g/cm³/% theory | g/cm³/% theory |
| 2a (2.) | 500° C., 10 h | 4.338/98.21 | 4.374/99.02 |
| 1a (1.) | 700° C., 1 h | 4.340/98.26 | 4.378/99.13 |
| 1b (1..) | 900° C., 0.1 h | 4.340/98.26 | 4.380/99.17 |
| 2b (2) | Green body | 4.338/98.21 | — |
| 3a | — | 4.340/98.26 | — |
| 3b | — | 4.340/98.26 | — |
| 4a | — | 4.337/98.18 | — |
| 4b | — | 4.337/98.18 | — |
| 5 (not | — | 4.324/97.91 | — |
| cut) | | | |

The results were examined and the following facts elucidated:

Green body: The titanium particles had been compressed to a very high density and surrounded the HA particles almost completely. No grain boundary pores were visible, or only very small ones. The titanium matrix appeared in principle as a dense material. The heat treatments at all of the tested temperature and time conditions had affected the microstructure and had probably caused the titanium particles to grow together, more significantly the higher the temperature used. The HA particles appeared unaffected at all the temperatures tested. However, a thin gap was observed between the titanium matrix and the HA particles of the heat-treated samples which seemed to increase with the temperature. At 500° C., the gap was scarcely visible (0-0.1 μm). At 700° C., it was found around the HA particles and was ca. 0.2 μm wide. At 900° C, the gap was more noticeable and was ca. 0.4 μm wide. The gap can still be considered small in view of the fact that the HA particles were ca. 100 μm in diameter and still held firm by surface irregularities and the tight-fitting titanium matrix.

A 98% compressed (unsintered) composite material of titanium powder and hydroxyapatite was produced by impact compaction.

The compression effect was observed throughout the sample body. The titanium matrix surrounded the HA particles.

The composite was heat-treated with the aim of binding the titanium particles to one another. The density increased to ca. 99%. The microstructure is already changed at 500° C., and more so at a higher temperature.

No reaction product between Ti and HA was observed visually in any of the samples, but a thin gap formed between the materials at high temperature. However, this gap was considered small in relation to the particle size of HA.

A presently proposed embodiment of a device, method and use will be described below with reference to the attached drawings in which

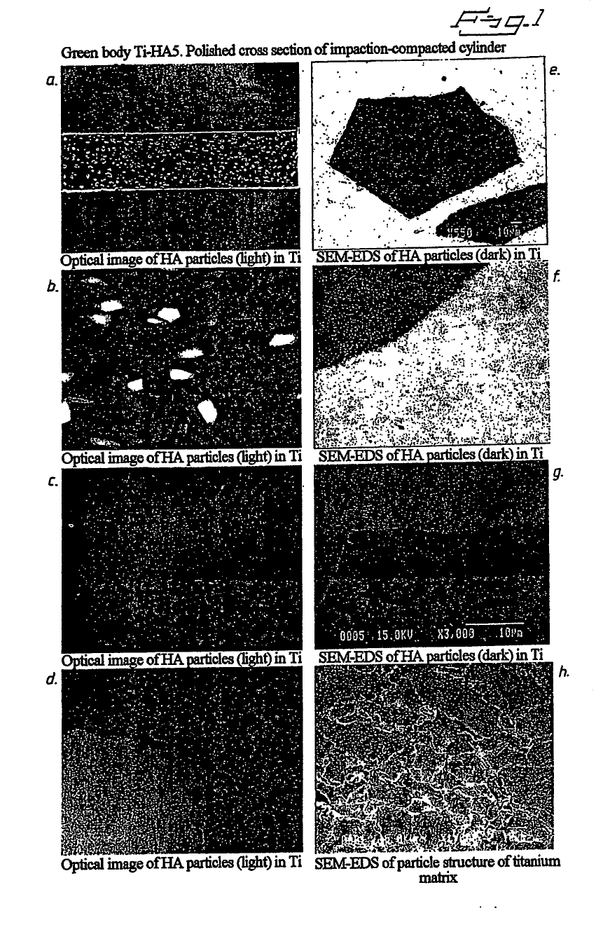

FIG. 1 shows, in different enlargements, the microstructure of composite material which has been compacted by impaction and has not thereafter been exposed to heat treatment,

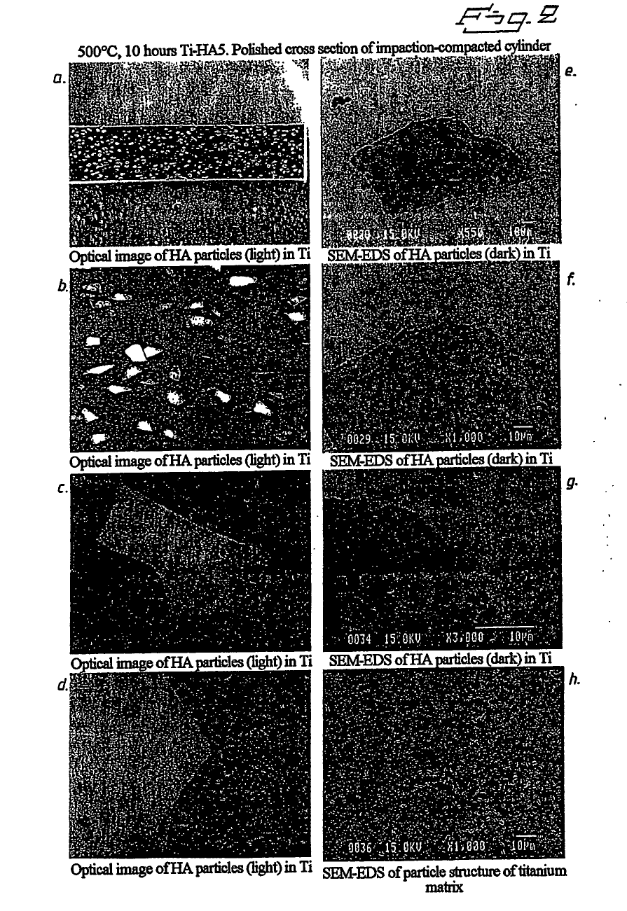

FIG. 2 shows, in different enlargements which correspond to the enlargements in FIG. 1, the microstructure of composite material which has been compacted by impaction and has thereafter been exposed to heat treatment at 500 degrees for 10 hours,

FIG. 3 shows, in a vertical view and diagrammatically, an implant in a jaw bone,

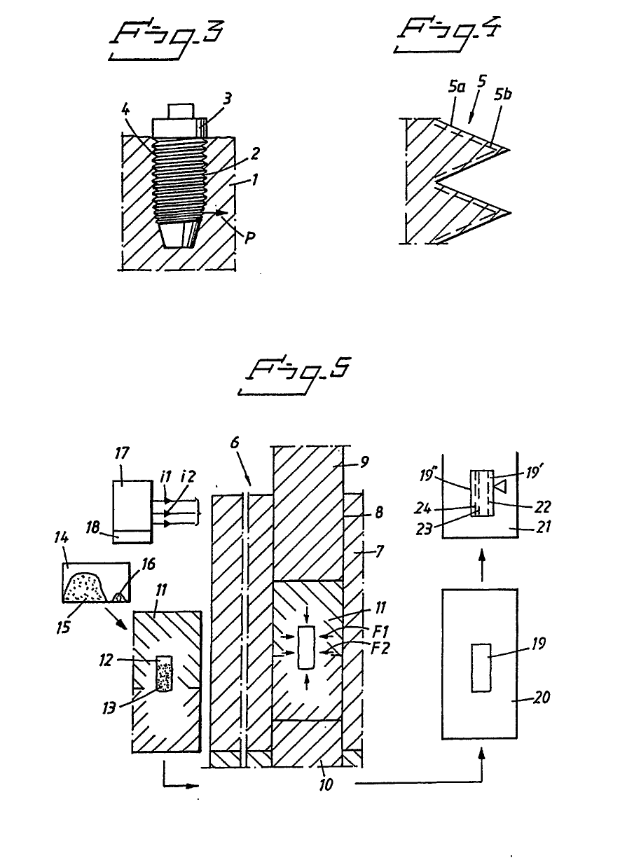

FIG. 4 shows, in a vertical view, parts of threads on an implant, and

FIG. 5 shows, in a vertical view and diagrammatically, a flow chart for production of a device in question.

FIG. 1 shows a microstructure of a green body Ti-HA5 with polished cross section of an impaction-compacted cylinder. The eight different subsidiary FIGS. a-h show different degrees of enlargement of HA particles applied in titanium in accordance with the above. The left-hand FIGS. a-d show optical images of HA particles in light configurations. FIGS. e-h show HA particles in dark configurations in the titanium. As will be seen from the figures, the titanium particles have been compressed to a very high density and surround the HA particles almost completely, except on the outside of the surface which is exposed to the bone or tissue in question. The HA particles are shown in different sizes and thus, for example, FIG. d shows the interface between a particle and the surrounding titanium. As can be seen from the figures, the HA particles can be considered as forming a pore system in the surface toward the bone or tissue. By means of this arrangement, a ragged outer surface can be considered to be present on the titanium body if the HA particles have come loose and have migrated over to the bone or tissue structure. This therefore increases the possibilities of secure incorporation of the implant or the like in the bone or tissue. The optical images are taken with a camera to show how the material looks (white particles in a metal matrix). The SEM-EDS images show the microstructure. On the SEM images, the HA particles are instead dark.

FIG. 2 shows corresponding enlargements of the microstructure in the composite material. In this case, the composite material has been heat-treated at 500° C. for 10 hours. For comparison of FIGS. 1 and 2, reference is made to the above analysis of results.

In FIG. 3, a jaw bone is indicated diagrammatically by 1. A hole or recess has been made in a manner known per se in the jaw bone to receive an implant 3 which can be of the type which has an external thread 4 by means of which the implant can be screwed into the hole 2. The implant can have a configuration already known per se and will therefore not be described in detail here.

FIG. 4 shows parts of a thread structure 5 which can be arranged on the implant 3 in FIG. 3. In accordance with the present invention, the actual outer surface 5a, or rather a part or portion 5b bearing the outer surface, is made of the composite material discussed above. The whole implant body or the outer surface(s) or portion(s) facing the bone 1 or tissue can be made of said composite material.

In FIG. 5, the impact-type compaction machine discussed above is indicated by 6. As the machine is well known per se, it will not be described in detail here, except to note that the machine comprises a die 7 which is provided with a recess 8 in which two stamps 9 and 10 can extend and in which an elastic mold 11 can be arranged. The mold made of elastic material is arranged to transmit the two-dimensional impact energy obtained via the stamps 9 and 10 to the powder mixture which can be placed in a diagrammatically indicated mold cavity 12 so as to give a three-dimensional product, for example said implant 3 according to FIG. 3. The powder mixture has been indicated by 13 in FIG. 5. The elastic mold is provided with punch members and mold cavity. The arrangement is moreover such that an isostatic function or isostatic action arises against the powder mixture, the result being that pressing forces, for example F1, F2, arise uniformly around the whole mold cavity and the powder mixture. In the present case, the stamps 9 and 10 operate toward and away from one another, with the mold 11 lying in between them. The internal punch arrangement of the mold is not shown in FIG. 5. The principles of this are shown in the Swedish patent application “Arrangement, device, method, product and use in connection with a blank made preferably of titanium powder and intended for a dental crown or other product for the human body” filed by the same Applicant on the same day as the present patent application. In a mixing unit 14, the titanium powder 15 and the HA powder 16 are mixed together in accordance with the above. The mixed-together powders are brought to the cavity 12 in the mold 11 and have been indicated by 13 in accordance with the above. The mold 11 comprises a top mold and a bottom mold which can be separated from one another and put together. The mold 11 with punch and powder is then transferred to the machine 6, of which one stamp 9, for example, can be removed from the recess 8 in order to allow the mold to be fitted. The machine is provided with a control unit 17 which can have a control panel 18. By means of the control unit, control signals 11 are generated for controlling the machine's movement/impact, kinetic energy, number of impacts, etc. When the machine's impacting unit is activated, the mold or molds 11 are acted upon so as to transfer the impact energy to the powder mixture and in this way create a blank/raw material. After the treatment or production in the machine 6, the raw material 19 is transferred to one or more subsequent treatment steps 20, 21, etc. In treatment step 20, the raw material 19 can be subjected to heat treatment, sintering, etc. In the treatment step, the heat-treated, sintered, etc., raw material 19′ can be subjected to chemical or mechanical working, for example turning, milling, shot-peening, electro-chemical treatment to obtain an oxide layer, etc. The raw blank 19′ which has been worked can then constitute an actual component, for example the component 3 in FIG. 3. In connection with the control of the machine by means of the control unit 17, control signals i2 can be established for producing different layers and/or positions of the HA particles so that at least some of these, preferably the majority of them, are exposed outward from their actual surface 19″ which is intended to face toward the actual bone or tissue. In FIG. 5, a number of layers of said type have been indicated by 22, 23 and 24. When the implant 3 is applied in the jaw bone (see FIG. 3), the HA particles or the HA fractions have the possibility of migrating into the surrounding bone depending on its composition.

In accordance with the invention, therefore, an impact-type compaction machine with a high impact compaction energy is used to compress the powder material and said agent in powder form to give a composite material which can form or be included in a component which can be fitted in a bone or a bone tissue in the human body. By means of the invention, it is possible to accelerate the incorporation of the implant or the like, without ignoring the long term. The titanium powder can have particle sizes of 20-50 μm (possibly up to 200 μm). The particles of HA can be given a cone shape and have sizes of 10-500 μm. Sintering temperatures of 100-1200° C. can be used.

The invention is not limited to the above embodiment, and instead it can be modified within the scope of the attached patent claims and the inventive concept.

1. A device which, via at least one surface or one portion, is arranged to be applied to bone and/or tissue in the human body, for example jaw bone, and which, at the surface or portion, is provided with an agent which stimulates bone growth, preferably HA (hydroxyapatite), where at least one surface-bearing part or the portion comprises or consists of compressed bone-compatible and/or tissue-compatible material, preferably titanium powder, characterized in that the powder material and the bone-growth-stimulating agent form a composite material which is obtained by means of impact compaction and, if appropriate, sintering.

2. The device as claimed in patent claim 1, characterized in that the bone-growth-stimulating/HA agent is arranged completely or partially in or at the actual surface layer and can thus be exposed to the bone and/or tissue in question.

3. The device as claimed in patent claim 1, characterized in that the bone-growth-stimulating agent is in the form of particulate fractions with sizes in the range of 90-120 μm.

4. The device as claimed in patent claim 1, characterized in that titanium powder with considerable purity, preferably a purity of 99.99%, and a relatively small particle size (Wah Chang HP (or CP) −325 Mesh T080014 (010607)) constitutes the base for the composite structure.

5. The device as claimed in claim 1, characterized in that titanium powder in a quantity of ca. 90-98%, preferably ca. 95%, and HA powder in a quantity of 2-10%, preferably 5%, form the starting material for the material compacted by impaction and possible sintering.

6. A method for producing a device, for example an implant, which, via at least one surface or one portion, is arranged to be applied to bone and/or tissue in the human body, for example jaw bone, and which, at the surface or portion, is provided with an agent which stimulates bone growth, preferably HA, where at least one surface-bearing part or the portion is made of compressed bone- compatible and/or tissue-compatible material, preferably titanium powder, the method comprising:
a) mixing together the bone-compatible and/or tissue- compatible powder material and said agent which is in powder form,
b) applying the mixture in a mold cavity belonging to a mold applied in a machine which effects impact compaction and which operates with a high impact compaction energy,
c) activating the impacting unit of the machine so that it acts on the mold and transfers the energy to the powder mixture and thereby creates a blank for the device,
d) treating the blank in one or more treatment units for producing the device from the blank.

7. The method as claimed in patent claim 6, characterized in that the blank is sintered and/or heat-treated and is subjected to chemical, electrochemical and/or mechanical treatment or machining (milling, turning, shot-peening, etc.).

8. The method as claimed in patent claim 6, characterized in that, in step a), titanium powder of considerable purity, for example 99.99%, and relatively small particle size is mixed together with HA, for example sintered HA, which has been crushed and screened to the fraction 90-120 μm.

9. The method as claimed in patent claim 8, characterized in that the mixture consists of ca. 95% titanium powder and 5% HA powder, and the powders are mixed in the dry state, with agitation and stirring.

10. The method as claimed in patent claim 8, characterized in that the machine is controlled so as to generate an impact compaction energy of ca. 335 Nm or higher and to execute one or more impacts against the mold.

11. The method as claimed in claim 6, characterized in that the titanium particles are compressed to a substantial density, for example 98%, and in that there is substantial surrounding of the HA particles.

12. The method as claimed in claim 6, characterized in that the positions of the HA particles in the composite material are controlled upon mixture and application in the mold cavity of the mold, and in that the blank is machined so that HA particles are present at the surface exposed to the bone and/or tissue.

13. Use in the production of a device made of compressible bone-compatible and/or tissue-compatible powder material, for example titanium powder, and provided with a bone-growth-stimulating agent, preferably HA, characterized in that an impact-type compaction machine with a high impact compaction energy is used to compress the powder material and said agent in powder form to give a composite material.
